(19) United States
(12) Patent Application Publication
(10) Pub. No.: US 2021/0345951 A1
(43) Pub. Date: Nov. 11, 2021

(54) Neuropathy Screening and Severity Instrument and System

(71) Applicant: University of Utah Research Foundation, Salt Lake City, UT (US)

(72) Inventors: Sayed Abdullah Sadat, Salt Lake City, UT (US); John Robinson Singleton, Salt Lake City, UT (US); Stormy Foster-Palmer, Salt Lake City, UT (US)

(21) Appl. No.: 17/308,824

(22) Filed: May 5, 2021

Related U.S. Application Data
(60) Provisional application No. 63/020,402, filed on May 5, 2020.

(51) Int. Cl. A61B 5/00; A61B 5/01; G16H 10/60

(52) U.S. Cl. CPC A61B 5/4827; A61B 5/0004; A61B 5/0022; A61B 5/01; A61B 5/7264; A61B 2562/0247; G16H 10/60; A61B 2560/02; A61B 2560/04; A61B 2562/0271; A61B 5/6801

(57) ABSTRACT

A neuropathy screening and severity system can comprise a contact probe comprising a probe housing having a predetermined weight and having an electromechanical actuator operable to vibrate at a variable amplitude and a frequency. The actuator can be operable to contact with a patient at a predetermined pressure based on the predetermined weight. The system can also comprise an electronic control unit disposed in the probe housing. The electronic control unit can be operable to ramp the variable amplitude of the electromechanical actuator from a first amplitude to a second amplitude over a sensory test time.

### RELATED APPLICATION

This application claims priority to U.S. Provisional Application No. 63/020,402, filed May 5, 2020 which is incorporated herein by reference.

### GOVERNMENT INTEREST

None.

### BACKGROUND

Peripheral nerves are bundles of nerve fibers, or axons, that reach from the spinal cord out to the face, trunk, and limbs to convey information about sensation, strength, and autonomic functions. Peripheral neuropathy is defined as a progressive, systemic injury to peripheral nerves, resulting in symptoms that are typically length-dependent and often symmetrical. This is distinguished from focal structural or vascular injuries to individual peripheral or cranial nerves that result in symptoms limited to the distribution of that single nerve, for instance, carpal tunnel syndrome due to compressive injury of the median nerve at the wrist, or lumbar radiculopathy, compressive injury to a single lumbar nerve root.

Frequently, peripheral neuropathy results from metabolic injury, for instance, due to diabetes, vitamin deficiencies, effects of medications and toxins, or as a consequence of infections, inappropriate immune activation, or cancer. Neuropathy caused by these diverse insults often shares similar symptomatic features, primarily distal loss of sensation, numbness, and tingling paresthesias, and neuropathic pain. As symptoms progress, sensory ataxia and weakness increases fall risk, pain can become disabling, and loss of protective sensation for foot injury predisposes patients to poorly healing wounds and infection that require prolonged treatment or amputation. Many forms of peripheral neuropathy have specific treatments that are more effective when provided early in the disease course. Early diagnosis is thus critical to effective treatment and avoids complications, helping patients deal with the disease and return to the maximum degree of normalcy in their daily lives.

Most peripheral neuropathy is first assessed and diagnosed in the Primary Care setting. Primary care diagnosis of length-dependent peripheral neuropathy is currently poorly sensitive. Although brief history questions can help practitioners to recognize patients with positive neuropathy symptoms (e.g., neuropathic pain, paresthesia), many patients with common neuropathic contributors, including metabolic syndrome or diabetes have neuropathic distal large fiber sensory loss that is asymptomatic but still places patients at risk for sensory ataxia, falls and injury. Recognition of neuropathy early in disease onset can prompt additional etiological testing and lifestyle modification. Sensation testing using a 10-gram monofilament at the great toe is currently the most common technique used in primary care foot screening for neuropathy. However, this testing protocol has remarkably poor sensitivity and only recognizes patients with moderate to severe disease. Timed vibration with a 128 Hz tuning fork is more sensitive but is technically difficult to deploy accurately. In general, sensory testing works best, and is most sensitive, when it moves anatomically from areas of absent sensation to areas of normal sensation (as with pin-sharp testing) or provides an increasing sensory stimulus that reaches a sensory detection threshold. Other clinical practice settings in which screening for peripheral neuropathy might be routine, or frequently indicated, include general Neurology, Physical Medicine and Rehabilitation, Sports Medicine, Rheumatology and Oncology.

A number of techniques and equipment can be employed to confirm if a patient suffers from neuropathy, and the extent of the neuropathy progression. These include nerve conduction studies, electromyography, nerve biopsy, and 3-millimeter punch biopsy of the skin to allow quantitation of intra-epidermal nerve fiber density. Often, confirmatory testing like nerve conduction studies are particularly valuable for secondary and differential diagnosis (i.e., what is the cause of the peripheral neuropathy). These confirmatory techniques require the use of specialized techniques, equipment, or access to reference pathology laboratories that are inappropriate for the primary care setting, or too expensive for primary care facilities to procure and use or maintain. Thus, simple, easily deployed, and economical techniques that can improve primary care screening for clinical peripheral neuropathy are needed and existing.

Separately, there is a need and clinical opportunity to reliably quantitate the severity of peripheral neuropathy in order to evaluate the progression of the disease and the effect of therapies. A casual examination of neuropathy severity is highly variable between examiners, limiting its use as a measure of progression. Confirmatory electrodiagnostic and biopsy techniques can be used to quantitatively follow disease progression but have the limitations of the expense and availability listed above.

### SUMMARY

A neuropathy screening and severity system can include a simple vibrational sensory screening unit, which includes a peripheral unit and a memory device. The memory device can be a dedicated processor, smartphone (i.e. installed application), tablet, desktop, or other computing device.

In one specific example, a neuropathy screening and severity system can comprise a contact probe comprising a probe housing having a predetermined weight and having an electromechanical actuator operable to vibrate at a variable amplitude and a frequency. The actuator can be operable to contact with a patient at a predetermined pressure based on the predetermined weight. The system can also comprise an electronic control unit disposed in the probe housing. The electronic control unit can be operable to ramp the variable amplitude of the electromechanical actuator from a first amplitude to a second amplitude over a sensory test time. The second amplitude can be higher than the first amplitude. This could be an exponential increment in steps. The power range for screening applications can vary from the power range for severity applications. The device can start in a non-vibrating state and a digital readout shows zero. As ramping begins, the digital readout increases accordingly. Once the vibration force increases to a level that the patient can feel, the sensory threshold, the output is noted from the digital readout. For use in a neuropathy severity examination, the electric power range is usually higher, and often begins from a nonzero starting point.

In some cases, the sensory test time (ramping time) can be from about 5 seconds to 60 seconds or more, and in some cases about 16 seconds.

In one example, the electromechanical actuator comprises a voice coil. Although power output can vary, in one example, the voice coil has an electric power output of 0.1 to 8 W, and optionally from 4 to 5 W. In some examples, the predetermined weight of the housing is within 10% of 0.66 pounds. In other examples, the predetermined pressure is from 2.5 to 8.0 pounds per square inch.

In some examples, the system is configured to include at least two amplitude control regimes including a coarse control and a fine control. The coarse control can span a large vibration amplitude ranged over a first sensory test time of 10-20 seconds. The fine control can span a variable amplitude range from a third amplitude to a fourth amplitude. The third amplitude can be 5% to 15% below the coarse response amplitude and the fourth amplitude can be 20% to 30% above the coarse response amplitude. The ramping can be repeated if the coarse response amplitude is not within about 5% of the fine response amplitude.

Although frequency can vary, non-limiting examples of suitable frequency is from 50 to 200 Hz. The frequency can be constant (i.e. fixed, non-user adjustable) or variable (i.e. user adjustable).

In one case, the electronic control unit is electrically connected to a mobile device via a wired connection. The mobile device can transmit control instructions to the electronic control unit, and the wired connection can provide power to the contact probe.

Alternatively, the electronic control unit can comprise a transceiver that is communicatively connected to a mobile device that transmits control instructions to the electronic control unit. The system can further comprise a battery operatively connected to the electronic control unit and the contact probe to power the electromechanical actuator.

In either wired or wireless options, the mobile device is often a smartphone.

In some cases, the input signal is a microphone signal (e.g. change in resistance). Optionally, a circuit connected as part of the peripheral unit can pre-process data from the memory device before transmission to the electromechanical actuator.

In order to improve consistency of test results, regardless of experience or user variability, the system can further comprise a pressure sensor oriented to measure an applied pressure of the contact probe against the patient.

Optionally, the system can further comprise a temperature sensor oriented to measure a patient temperature.

In another option, the system can further comprise an amplifier circuit adapted to increase input power to a target output power to the electromechanical actuator.

A method for conducting neuropathy screening is also provided herein. The method can include applying a contact probe to a toe of a patient with a predetermined pressure created by a weight of the contact probe, and activating an electromechanical actuator at a first amplitude at a predetermined frequency. Once activated, an amplitude of the electromechanical actuator can be increased from the first amplitude until the patient indicates that the patient can feel vibration from the electromechanical actuator. The amplitude at which the patient felt the vibration can be recorded.

In one option, the increasing step comprises incremental increases of the amplitude. Alternatively, the increasing step can comprise continuously increasing the amplitude. The amplitude can be increased linearly or parabolically. In some instances, the amplitude can be increased based on a machine learning algorithm and a patient's biometric information. The first amplitude can also be based on a machine learning algorithm and a patient's biometric information.

The contact probe can be controlled via a mobile device communicatively connected to the contact probe. The amplitude at which the patient first felt the vibration can be recorded in a database and can be correlated to the patient's age, sex, and known medical conditions. The initial amplitude used for ramped amplitude testing can be set based on at least one of the patient's age, sex, and known medical conditions.

The increasing step can comprise at least two amplitude control regimes including a coarse control and a fine control to produce a coarse response amplitude and a fine response amplitude from the patient. The coarse control can span a first sensory test time of 10-20 seconds. The fine control can span a variable amplitude range from a third amplitude to a fourth amplitude. The third amplitude can be 5% to 15% below the coarse response amplitude and the fourth amplitude can be 20% to 30% above the coarse response amplitude. The increasing step can be repeated if the coarse response amplitude is not within about 5% of the fine response amplitude.

In another example, a neuropathy screening and severity system is provided. The system can comprise a contact probe comprising a probe housing having a predetermined weight and having an electromechanical actuator operable to vibrate at a variable amplitude and a frequency. The actuator can be operable to contact with a patient at a predetermined pressure based on the predetermined weight.

The system can further comprise a pressure sensor disposed on the probe housing operable to verify the predetermined pressure, and an electronic that is operable to ramp the variable amplitude from a first amplitude to a second amplitude over a sensory test time. The system can further comprise an artificially intelligent database comprising a non-transitory storage medium, such as a memory, storing information correlating populations of patients to amplitudes at which patients feel vibrations from the contact probe, the first amplitude being based on the information stored in the database. The populations of patients can include the age, sex, and medical conditions of the patients. In this example, the contact probe does not test for any other sensory modalities besides pressure.

This system and method can be particularly adapted to assessing diabetic neuropathy, as well as potentially other forms of neuropathies which involve a sensory component.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

FIG. 1 shows a schematic view of an exemplary neuropathy screening and severity system.

FIG. 2A shows an exemplary contact probe for use in a neuropathy screening and severity system; FIG. 2B shows a schematic view of the contact probe of FIG. 2A, and FIG. 2C shows an exploded view of the contact probe of FIG. 2A.

FIG. 3 shows a schematic view of an exemplary mobile device for use in a neuropathy screening and severity system.

FIG. 4 shows a schematic view of an exemplary artificial intelligence dedicated system for use in a neuropathy screening and severity system.

FIG. 5 shows an exemplary method of conducting neuropathy screening using a neuropathy screening and severity system.

These drawings are provided to illustrate various aspects of the invention and are not intended to be limiting of the scope in terms of dimensions, materials, configurations, arrangements or proportions unless otherwise limited by the claims.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

Definitions

In describing and claiming the present invention, the following terminology will be used.

The singular forms “a,” “an,” and “the” include plural referents unless the context clearly dictates otherwise. Thus, for example, reference to “a sensor” includes reference to one or more of such devices, and reference to “subjecting” refers to one or more such steps.

As used herein, the term “about” is used to provide flexibility and imprecision associated with a given term, metric or value. The degree of flexibility for a particular variable can be readily determined by one skilled in the art. However, unless otherwise enunciated, the term “about” generally connotes flexibility of less than 2%, and most often less than 1%, and in some cases less than 0.01%.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the term “at least one of” is intended to be synonymous with “one or more of.” For example, “at least one of A, B and C” explicitly includes only A, only B, only

C, and combinations of each.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

Neuropathy Screening and Severity Systems

One of the advantages of this solution is its increased usability. Other neuropathy screening or severity methods in primary care settings with medical assistants have demonstrated the difficulty that non-neurologists have with multi-step screening or severity methods. Additionally, this solution offers the following advantages over existing solutions. The solution is fast compared to non-electronic solutions. The solution employs minimum mechanical and electronic parts, therefore, minimizing the mechanical, electrical losses, and/or attenuation of the signal associated with each additional mechanical or electronic parts. Minimum noise and minimum mechanical and electronic parts contribute to the increase in the accuracy. In addition, the smartphone/computer application version of the solution produces a high-quality wave-shape such as but not limited to sinusoidal wave-shape, generated compared to the existing solutions. The app-based version used to operate the peripheral device can be easily programmed to suit any algorithm without the need to modify the electronic circuits or any mechanical part on the contact probe. This characteristic makes the solution more versatile than any other available solution. The simplicity of the solution (minimal mechanical and electronic parts) makes the device case and weight flexible enough to create a highly ergonomic design and easy to hold in hand.

The device will also improve inter-rater reliability. Practitioners will not need to precisely replicate 10 gram monofilament testing algorithms, or alternatively will not have to hold a tuning fork to the patient's toe for a prolonged period of time, and the pressure can be controlled by the device, rather than the practitioner. The user interface display of the smartphone/computer can be more convenient, more informative, easy to use, and can include additional features. Additionally, the simplicity of the solution makes it highly portable and its superior user-friendliness would require the minimum skill or no extra instructions to be operated (other than the instructions that can be embedded in the app). Due to the minimum use of mechanical and electronic components, it has less vulnerabilities to defects, breaks, and malfunctions.

The system can produce ascending vibration amplitude at a constant frequency of 128 Hertz but changing amplitude from low to high. The system can also produce descending vibration amplitude and a different frequency value if needed. The system can adjust and scale different amplitude ranges to the scale of (0-100). One or more pressure sensors that can optionally be incorporated into the system can help in ensuring that the pressure applied during each screening or severity is identical and reproducible. The optional temperature sensors in the system can be used to measure the temperature of the patients' skin if the temperature factor is desired to be considered in calculating the neuropathy index. The system can provide a numerical read out (neuropathy index) which will be used to assess degree of sensation loss.

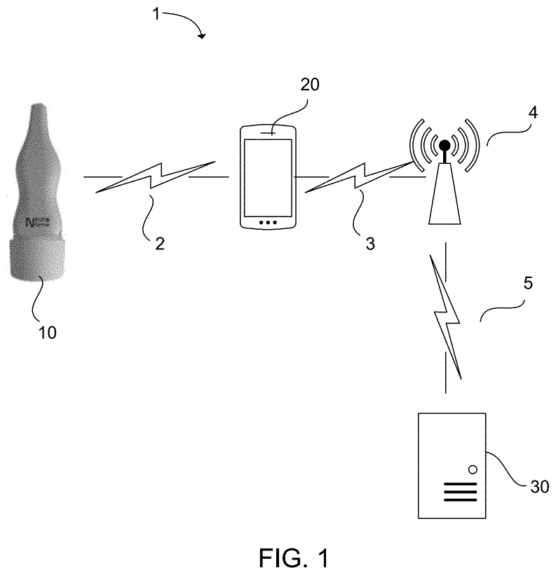

Referring now to the figures, a neuropathy screening and severity system is schematically shown in FIG. 1. The neuropathy screening and severity system 1 can comprise a neuropathy screening contact probe 10 operable to aid a practitioner in diagnosing the presence and/or severity of peripheral neuropathy. The neuropathy screening contact probe 10 can be communicatively connected to mobile device 20. For example, the neuropathy screening contact probe 10 can be wirelessly connected to the mobile device 20 via a short-range wireless communication protocol 2 such as Bluetooth or near field communication (NFC). In some examples, the neuropathy screening contact probe 10 can connect to the mobile device 20 via a wired connection, such as a standard 3.5 mm audio cable, USB A, USB C, mini-USB, Thunderbolt interface, lightning connector, and the like.

The mobile device 20 can connect to an artificially intelligent dedicated system 30. The mobile device 20 can connect to the system 30 such as via a network 5. The network 5 can be a local network such as a LAN or WAN protocol. In some examples, the network connection 5 can be the Internet. The mobile device 20 can connect to the network 5 via a wireless access point 4 through a wireless or wired connection 3 such as via a wireless connection using Wi-Fi. The mobile device 20 can be operable to control one or more functionalities of the contact probe 10. The mobile device 20 can also communicate with the artificially intelligent dedicated system 30 to report information entered at the mobile device 20 and/or feedback received from the contact probe. The mobile device 20 can further receive information from the artificially intelligent dedicated system 30 to alter control instructions sent to the contact probe 10. In one example, the mobile device system will also perform the functions artificially intelligent dedicated system 30. Thus, in some examples, the system includes the contact probe 10 and mobile device can operate as a standalone system without the artificially intelligent dedicated system 30. In this case, the control unit is directed by the instructions and signals received from the mobile device 20. Alternatively, the control unit can include a memory device and CPU in which the instructions are integrated into the handheld system as a single unit which does not need an external processor (e.g. mobile device). In this case, the instructions are programmed directly into the electronic control unit. Further, the integrated unit can also optionally include the artificially intelligent dedicated system 30.

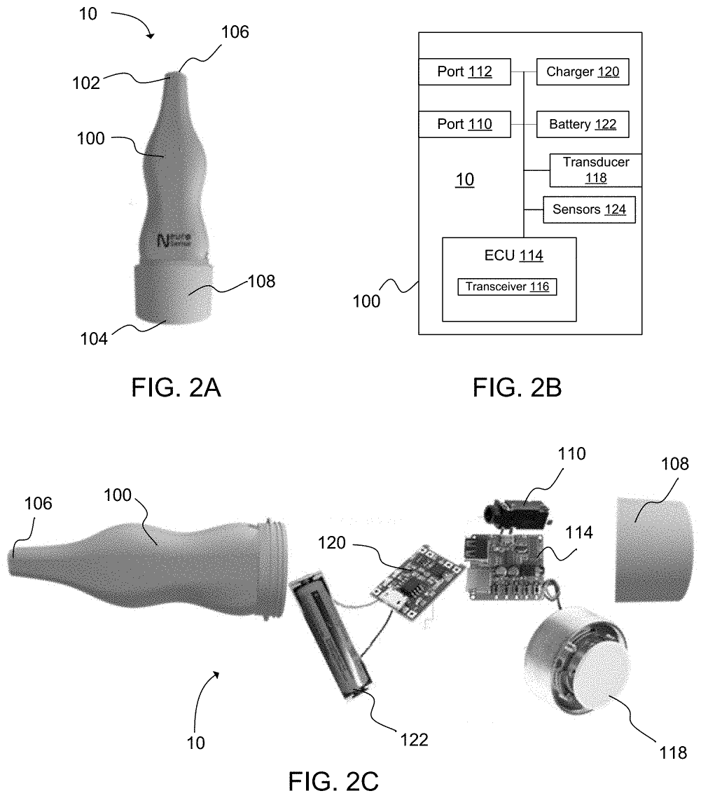

FIGS. 2A-2C show an exemplary contact probe for use in a neuropathy screening and severity system. The contact probe 10 comprises a probe housing 100. The probe housing 100 can be formed in an ergonomic shape to facilitate easy gripping and use by a practitioner. The probe housing 100 can be formed from any suitable material such as a polymer base material that is injection molded to form the housing 100. Of course, other materials and manufacturing methods to construct the housing 100 can also be utilized.

The probe housing 100 can comprise a first end 102 and a second end 104 opposite the first end 102. A probe tip 106 can be disposed at the first end 102 of the housing 100. The probe tip 106 can be configured to contact a patient during use. For example, the probe tip 106 can be applied to a toe of a patient during use. In some examples, the probe tip 106 is applied to a great toe of the patient. In some examples, the probe tip 106 is applied to the dorsum of a toe (e.g. to the dorsal side of the proximal or distal interphalangeal joint of the toe).

The probe housing 100 can further comprise a removable cap 108 disposed at the second end 104 of the housing 100. The removable cap 104 can be selectively attached to and detached from the housing 100 via a threaded connection or via any other suitable mechanism. The removable cap provides access to the interior of the housing 100.

The probe housing 100 can comprise one or more ports 110, 112 disposed on an exterior of the housing. For example, a port 110 can comprise a 3.5 mm port to receive a standard 3.5 mm audio cable to connect the contact probe 10 to an external device, or other suitable data connection (e.g. USB A, USB C, mini-USB, Thunderbolt interfaces, lightning connector, and the like). A port 112 can comprise a charge port, such as a USB or other similar port to connect the contact probe 10 to a power source. In some examples, the contact probe 10 can comprise a battery 122 disposed with the housing 100. The battery 122 provides power to the contact probe during use. The battery 122 can be a rechargeable battery that is charged when the port 112 is connected to an external power supply. The contact probe 10 can comprise a charge controller 120 that controls a flow of electricity into the battery 122.

The contact probe 10 can further comprise an electronic control unit (ECU) that is operable to control one or more functionalities of the contact probe 10. For example, the ECU can comprise one or more transceivers 116 that are operable to communicatively connect to an external device via a short range wireless communication protocol, such as Bluetooth or NFC. The ECU can be connected to a transducer 118. The transducer 118 can comprise an electromechanical actuator capable of vibrating at a variable amplitude and frequency. For example, the electromechanical actuator of the transducer 118 can comprise a voice coil or a piezoelectric element to produce vibrations. The voice coil in this example can have an electric power output of 0.1 to 8 W. In some examples, the electric power output can range from 4 to 5 W. In one example, the transducer 118 can vibrate at frequencies ranging from 50 Hz to 200 Hz. In some examples, the vibration can be set at a fixed frequency (e.g. 128 Hz). In some examples the vibration can be variable. In some examples, the frequency can be provided in a sinusoidal pattern. In some examples, the transducer comprises an amplifier circuit adapted to increase input power to a target output power to the electromechanical actuator. The ECU 114 can operate the transducer 118 based on instructions received from an external device (such as the mobile device 20 in FIG. 1) via the transceiver 116.

The transducer 118 can be disposed in the tip 102 of the housing 100, such protruding from the tip 102 of the housing 100 to transmit vibrations to a patient during use. In some embodiments, the transducer is mounted in the housing 100 to cause the entire housing 100 to vibrate such that the tip 102 transmits vibrations to the patient during use.

In some embodiments, the contact probe 10 can comprise other sensors 124. Such sensors 124 can comprise a pressure transducer that provides a practitioner feedback such that the practitioner can know how much pressure is applied to the patient. The sensors 124 can also comprise a thermometer to measure temperature of the patient at the contact point.

The housing 100 can be formed to have a predetermined standard weight. This allows a desired pressure to be applied to a patient using only the weight of the contact probe 10. In this manner, the pressure applied to patients is substantially consistent even when the contact probe 10 is used by different practitioners. The housing 100 can be formed with a thickness or pattern to give it the desired weight. In some embodiments, the housing 100 can comprise a separate weight that is disposed within the housing 100 such that the contact probe 10 has the desired predetermined weight. In some examples, the predetermined weight is within 10% of 0.66 pounds.

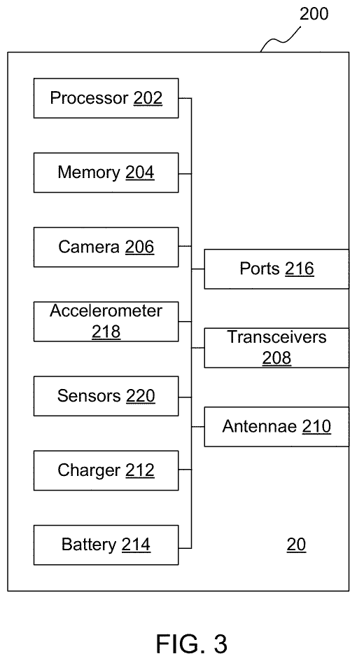

As mentioned above, the contact probe 10 can communicatively connect with a mobile device 20. FIG. 3 shows an exemplary mobile device for use with a neuropathy screening and severity system. A mobile device 20 can be any suitable mobile computing device such as a mobile phone (i.e. smartphone), a tablet computer, or the like. The mobile device 20 can be considered a “remote device” that separately controls the contact probe. The mobile device 20 comprises a mobile device housing 200. The housing 200 can comprise a screen such as a touchscreen for providing visual outputs and receiving inputs for the mobile device 20. The housing 200 can comprise a processor 202 that controls functionality of the mobile device. For example, the processor 202 can execute machine readable instructions to operate processes as set forth in an operating system, application software, and the like. The mobile device 20 can further comprise a memory 204 which stores data in including operating system data, application data, document data, etc.

The mobile device 20 can comprise a camera 206 as another input device to capture still and video images, as well as to scan one or two dimensional barcodes, and/or to provide other input to the mobile device 20 by capturing visible, infrared, and/or ultraviolet light. The mobile device 20 can further comprise one or more transceiver 208 connected to antennae 210. The transceivers 208 can operate according to any desired communications protocols such as Bluetooth, NFC, or Wi-Fi. As mentioned above, the mobile device 20 can connect to the contact probe 10 via a short-range wireless communication protocol 2 (see FIG. 1) such as Bluetooth. The mobile device 20 can also connect to a wireless access point 4 via a wireless network 3.

The mobile device 20 can comprise a charge controller 212 and battery 214 to provide power to the mobile device 20. The charge controller 212 can control the flow of electricity to the battery 214 to control the state of charge of the battery 214. The battery 214 and charge controller can be connected to a one of ports 216 that can receive a cable adapter to provide power to recharge the battery 214. The ports 216 can comprise a USB or other standard or proprietary port to connect to a power source or to transmit data to and from the mobile device 20. The ports 216 can also comprise a 3.5 mm audio port to receive a standard 3.5 mm audio wire. This can facilitate a wired connection with the contact probe 10 as an alternative to the wireless connection.

The mobile device 20 can comprise other features and components. For example, the mobile device 20 can comprise an accelerometer 218 and other sensors 220 such as a magnetometer, a gyroscope, a fingerprint sensor, or the like.

Referring to FIGS. 1-3, the mobile device 20 can be operable to run an application that can be downloaded and stored in a memory 204 of the mobile device 20 to control the contact probe 10. For example, the mobile device 20 can send control instructions to the contact to probe 10 to cause the ECU 114 of the contact probe 10 to operate the transducer 118 to cause the electromagnetic actuator to vibrate at a certain frequency and at a certain amplitude. The mobile device 20 can cause the frequency and amplitude to change based on input to the mobile device 20 or based on the program operating on the mobile device 20. The mobile device 20 can receive feedback from the contact probe 10 such as confirmation of the operating state of the transducer 118 or information from one or more sensors 124 such as for applied pressure or temperature.

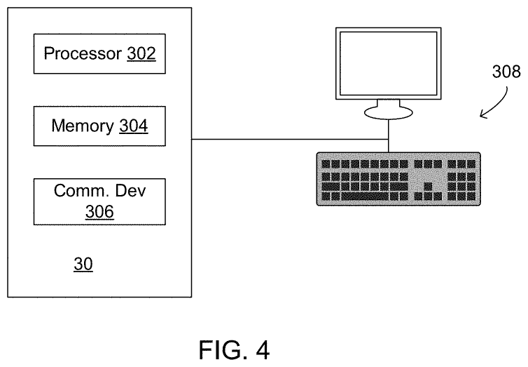

The mobile device 20 can be communicatively connected to an artificially intelligent dedicated system 30. FIG. 4 shows a schematic of an exemplary artificially intelligent dedicated system. The artificially intelligent dedicated system 30 can be hosted on a server that comprises a processor 302, a memory 304, and a communications device 306. The communications device 306 can be any suitable networking device operable to communicate via the network 5 (see FIG. 1). The artificially intelligent dedicated system 30 can comprise peripheral devices 308 such as a monitor and keyboard to receive inputs to and provide outputs from the artificially intelligent dedicated system 30.

The artificially intelligent dedicated system 30 can connect to a plurality of mobile devices, such as mobile device 20 to receive information from the mobile devices and to send information to the mobile devices. The artificially intelligent dedicated system 30 can host an artificially intelligent database comprising information received from the various mobile devices.

In this example, artificially intelligent dedicated system 30 can host a database with information for the neuropathy screening and severity system. For example, the artificially intelligent dedicated system 30 can receive information from the mobile devices that comprises patient information entered at the mobile device 20 and screening information based on diagnosis data obtained via the contact probe 10.

In some examples, the artificially intelligent dedicated system 30 can receive biometric information corresponding to a patient profile. The biometric information can be tied to a specific patient or the biometric information can be anonymous. The biometric information can comprise a patient's, age, sex, race, weight, height, blood pressure, temperature, current medical conditions, previous medical conditions, and the like. The biometric information can be correlated with a patient's neuropathy screening results obtained by practitioners using the contact probe 10. The screening results can comprise a threshold frequency and amplitude at which a patient can feel the applied vibrations of the contact probe.

In some examples, the artificially intelligent dedicated system 30 can receive a pre-test assessment, recorded in the application by the testing practitioner using a forced choice set of check boxes, about whether a neuropathy is not suspected clinically, suspected, or present for the patient to be screened.

The artificially intelligent dedicated system 30 can be operable to execute machine learning algorithms on the data stored in the artificially intelligent database of the artificially intelligent dedicated system. By correlating the biometric information, pretest probability of a clinical neuropathy diagnosis and screening results from a plurality of patients, the artificially intelligent dedicated system 30 can develop baselines or expected neuropathy results for patients having certain biometric characteristics. This can aid a practitioner in screening a new patient by understanding what frequencies and amplitudes of vibrations of the contact probe 10 are normal for the biometric characteristics. Further, the artificially intelligent dedicated system 30 can provide feedback to a practitioner as to whether screening results for a particular patient fall within normal thresholds for the patient's biometric characteristics, and/or whether a patient's neuropathy is progressing over time better or worse than expected.

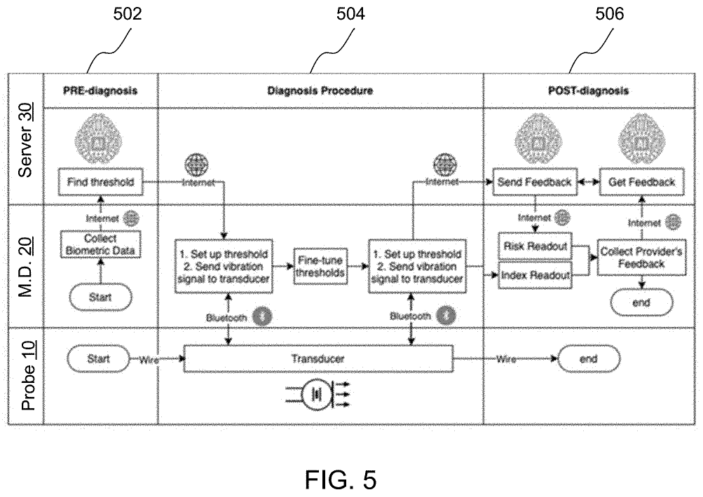

FIG. 5 shows an exemplary method of conducting neuropathy screening using a neuropathy screening and severity system. In step 502 during pre-diagnostic, a practitioner using a mobile device 20 with an application operable to control a contact probe 10, collects biometric data from a patient. The biometric data can include the patient's age, sex, race, weight, blood pressure, temperature, and medical condition(s). The biometric data obtained at the mobile device 20 can be sent to the artificially intelligent dedicated system 30 via the network 5 (see FIG. 1), such as the Internet. The practitioner can prepare the contact probe 10 and place the contact probe 10 on the patient, such as on the dorsum of the great toe. The weight of the contact probe 10 provides the correct predetermined pressure to the dorsum of the great toe for diagnosis. The transducer 118 of the contact probe 10 can be activated via the application running on the mobile device 20.

In step 504, the practitioner diagnoses the patient to determine the presence and severity of neuropathy. The artificially intelligent dedicated system 30 can send a threshold or baseline amplitude and/or frequency to the mobile device 20 based on the biometric data collected at the mobile device 20 by the practitioner. By using the threshold range or baseline amplitude generated by the artificially intelligent dedicated system 30, accuracy of the screen can be increased, while the total diagnostic time can be reduced, which can be advantageous for busy practitioners. The threshold or baseline amplitude and/or frequency can be applied to the patient via the transducer 118 of the contact probe 10 via instructions sent to the contact probe 10 via the mobile device 20. Based on feedback from the patient, the practitioner can fine-tune the amplitude and/or frequency of the vibration via inputs to the mobile device.

The fine-tuning of the amplitude and/or frequency can comprise starting from a threshold or baseline amplitude based on the biometric data and increasing or ramping the amplitude until a response amplitude is found where the patient indicates that the patient can feel the vibration. In some examples, the amplitude and the frequency can be varied during fine-tuning. In some examples, the frequency remains fixed and only the amplitude is varied during fine-tuning. The increase or ramping in amplitude can be done in a variety of ways. For example, the amplitude can be increased using different patterns such as a linear or in a parabolic progression. The increasing or ramping of the amplitude can be repeated at different frequencies, can be repeated at the same or different rates, and can be repeated at the same or at a different ramping pattern. The increasing or ramping of amplitude can be based on information received from the artificially intelligent dedicated system 30 based on the patient's biometric information.

The fine-tuning of the amplitude and/or frequency of the vibration can be done in both a coarse control and a fine control to produce a coarse response amplitude and a fine response amplitude. The course control can span a first sensory test time of 10-20 seconds to determine the coarse response amplitude at which the patient can feel the vibration. The fine control can span a variable amplitude range from about 5% to 15% below the coarse response amplitude to 20% to 30% above the course response amplitude to determine the fine response amplitude. In some instances, the ramping or increase in the amplitude can be repeated if the coarse response amplitude is not within about 5% of the fine response amplitude.

The coarse control stage is aimed to find the range of vibration for the patient at which the patient feels the vibration. The fine control stage is aimed to find a precise and accurate value of the vibration amplitude where the patient started feeling it, and a neuropathy index can be calculated from this value of the vibration amplitude. The number of the tests in the fine control stage can be one or more depending on the desired value of accuracy and precision. In this way, more accurate neuropathy screening results can be achieved as compared to prior methods.

When a response amplitude has been found via fine-tuning the amplitude and/or frequency of the transducer, the mobile device 20 can instruct the contact probe 10 to deactivate the transducer, and the mobile device 20 can record the response amplitude. The response amplitude can be sent via the network 5 to the artificially intelligent dedicated system 30, and linked to biometric data.

Step 506 comprises a post-diagnosis operation of the neuropathy screening and severity system. The artificially intelligent dedicated system 30 can send feedback to the mobile device 20 via the network 5 concerning the results of the screening based on the response amplitude reported by the mobile device 20 and the patient's biometric information. For example, the artificially intelligent dedicated system 30 can determine whether the patient's response amplitude falls within a normal range based on the patient's biometric information. Further, the artificially intelligent dedicated system 30 can track the recovery from or progression of neuropathy in a particular patient over time. The response amplitude received at the artificially intelligent dedicated system 30 can also be incorporated into the artificially intelligent database along with the practitioner's reported pretest probability of a clinical diagnosis of peripheral neuropathy, and any other practitioner feedback sent via the mobile device 20. These results can further refine the machine learning algorithms at the artificially intelligent dedicated system 30 to provide better and more relevant information to practitioners during later uses of the neuropathy screening and severity system. For example, the artificially intelligent dedicated system 30 using machine learning can better estimate thresholds amplitudes for later patients as well as more accurately plan testing algorithms for later patients.

Many modifications can be implemented into the above described system and method without departing from the disclosure presented herein. For example, in one alternative, a wired self-contained controller (e.g. a microcontroller-based signal generator and control mechanism with display and controllers) can directly attach to the transducer wirelessly or through a wire. This also can include the wire directly attached or through other connection mechanism such as but not limited to the audio jack.

In another alternative, a wireless self-contained controller (e.g. a microcontroller-based signal generator and control mechanism with display and controllers) can be connected to the transducer through wireless connection methods such as but not limited to Bluetooth.

Through control of the contact probe by the application and based on the artificially intelligent features, the system also offers the potential to extend the technology's use and customers from the primary care or other medical providers to patients at home. In other words, with these feature, patients and people at risk of neuropathy can directly use the technology with zero to minimum expertise of neuropathy diagnosis. Furthermore, the artificially intelligent dedicated system 30 can also provide statistical information from the interaction of the technology with its users. The contributions of the artificially intelligent dedicated system 30 make the technology impressively smart while preserving its simplicity.

Each of these alternatives can have none, one, or more of the following features, power enhancement/amplitude boost mechanisms, pressure sensors, and temperature sensors.

In some embodiments, the contact probe can further comprise a processor, a memory, and one or more input and output devices such as buttons, speakers, LCD panels, and the like to allow a user to interact with the contact probe. In this instance, the contact probe can run an application directly and communicate directly with the artificially intelligent dedicated system. In this variation, the mobile device can be omitted from the system.

The foregoing detailed description describes the invention with reference to specific exemplary embodiments. However, it will be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

What is claimed is:

1. A neuropathy screening and severity system comprising,
a contact probe comprising a probe housing having a predetermined weight and having an electromechanical actuator operable to vibrate at a variable amplitude and a frequency, said actuator operable to contact with a patient at a predetermined pressure based on the predetermined weight; and
an electronic control unit disposed in the probe housing, the electronic control unit operable to ramp the variable amplitude of the electromechanical actuator from a first amplitude to a second amplitude over a sensory test time.

2. The system of claim 1, wherein the electromechanical actuator comprises a voice coil or a piezoelectric element.

3. The system of claim 2, wherein the voice coil has an electric power output of 0.1 to 8 W.

4. The system of claim 1, wherein the predetermined weight is within 10% of 0.66 pounds.

5. The system of claim 1, wherein the predetermined pressure is from 2.5 to 8.0 pounds per square inch.

6. The system of claim 1, wherein the system is configured to include at least two amplitude control regimes including a coarse control and a fine control to produce a coarse response amplitude and a fine response amplitude from the patient.

7. The system of claim 6, wherein the coarse control spans a first sensory test time of 10-20 seconds.

8. The system of claim 6, wherein the fine control spans a variable amplitude range from a third amplitude to a fourth amplitude, wherein the third amplitude is 5% to 15% below the coarse response amplitude and the fourth amplitude is 20% to 30% above the coarse response amplitude.

9. The system of claim 6, wherein the ramping is repeated if the coarse response amplitude is not within about 5% of the fine response amplitude.

10. The system of claim 1, wherein the frequency is from 50 to 200 Hz.

11. The system of claim 1, wherein the frequency is constant.

12. The system of claim 1, wherein the electronic control unit is adapted to wirelessly connect to a mobile device.

13. The system of claim 1, further comprising at least one of:
a pressure sensor oriented to measure an applied pressure of the contact probe against the patient;
a temperature sensor oriented to measure a patient temperature; and
an amplifier circuit adapted to increase input power to a target output power to the electromechanical actuator.

14. A method for conducting neuropathy screening comprising:
applying a contact probe to a toe of a patient with a predetermined pressure created by a weight of the contact probe;
activating an electromechanical actuator at a first amplitude at a predetermined frequency;
increasing an amplitude of the electromechanical actuator from the first amplitude until the patient indicates that the patient can feel vibration from the electromechanical actuator; and
recording the amplitude at which the patient felt the vibration.

15. The method of claim 14, wherein the increasing step comprises at least one of:
a) incremental increases of the amplitude;
b) continuously increasing the amplitude;
c) increasing the amplitude linearly;
d) increasing the amplitude parabolically; and
e) based on a machine learning algorithm and a patient's biometric information.

16. The method of claim 14, wherein the first amplitude is based on a machine learning algorithm and a patient's biometric information.

17. The method of claim 14, wherein the contact probe is controlled via a mobile device communicatively connected to the contact probe.

18. The method of claim 14, wherein the amplitude at which the patient felt the vibration is recorded in a database and is correlated to the patient's age, sex, and known medical conditions.

19. The method of claim 14, wherein the first amplitude is set based on at least one of the patient's age, sex, and known medical conditions.

20. The method of claim 14, wherein the increasing step comprises at least two amplitude control regimes including a coarse control and a fine control to produce a coarse response amplitude and a fine response amplitude from the patient.

21. The method of claim 20, wherein the fine control spans a variable amplitude range from a third amplitude to a fourth amplitude, wherein the third amplitude is 5% to 15% below the coarse response amplitude and the fourth amplitude is 20% to 30% above the coarse response amplitude.

22. The method of claim 20, wherein the increasing step is repeated if the coarse response amplitude is not within about 5% of the fine response amplitude.

23. A neuropathy screening and severity system comprising,
a contact probe comprising a probe housing having a predetermined weight and having an electromechanical actuator operable to vibrate at a variable amplitude and a frequency, said actuator operable to contact with a patient at a predetermined pressure based on the predetermined weight;
a pressure sensor disposed one the probe housing operable to verify the predetermined pressure;
an electronic control unit that is operable to cause the system to ramp the variable amplitude from a first amplitude to a second amplitude over a sensory test time;
an artificially intelligent database comprising a non-transitory storage medium storing information correlating populations of patients to amplitudes at which patients feel vibrations from the contact probe, the first amplitude being based on the information stored in the database.

24. The neuropathy screening and severity system of claim 23, wherein the populations of patients include the age, sex, height, weight, and medical conditions of the patients.

25. The neuropathy screening and severity system of claim 23, wherein the contact probe does not test for any other sensory modalities besides pressure.
